(19) United States
(12) Patent Application Publication
(10) Pub. No.: US 2021/0369243 A1
(43) Pub. Date: Dec. 2, 2021

(54) SYSTEMS AND METHODS FOR ESTIMATING CARDIAC FUNCTION AND PROVIDING CARDIAC DIAGNOSES

(71) Applicant: Dyad Medical, Inc., Allston, MA (US)

(72) Inventors: Hao ZHOU, Malden, MA (US); Ronny SHALEV, Brookline, MA (US)

(21) Appl. No.: 16/937,750

(22) Filed: Jul. 24, 2020

Related U.S. Application Data
(60) Provisional application No. 63/032,440, filed on May 29, 2020.

(51) Int. Cl. A61B 8/08; A61B 8/06; A61B 8/00; G16H 50/20

(52) U.S. Cl. CPC A61B 8/5223; A61B 8/065; A61B 8/0883; G16H 50/20; A61B 8/461

(57) ABSTRACT

A system and methods automatically predict left ventricular ejection fraction by processing echocardiogram data performed by software executed on a computer system. One example method includes inputting echocardiogram data from an echocardiogram device, identifying two-halves left ventricle segmentation based on the echocardiogram data for each time frame, estimating a left ventricular volume or area from the two-halves left ventricle segmentations for the each time frame, detecting end-diastolic states and end-systolic states by comparing the left ventricular volumes or area of the each time frames automatically with a moving window, and predicting a left ventricular ejection fraction based on said end-systolic states and end-diastolic states. A prognosis or treatment plan may be provided based on the left ventricular ejection fraction calculated.

### FIELD OF THE INVENTION

The invention relates systems and methods of estimating cardiac function and providing cardiac diagnoses using deep learning-based echocardiogram interpretation. More specifically, the invention combines a deep learning U-Net model with classical echocardiographic assessment methods to predict the left ventricular ejection fraction in real time.

### BACKGROUND OF THE INVENTION

An echocardiogram is a common and useful ultrasound imaging technique used for viewing a patient's cardiac function. It is estimated that there are over ten million echocardiograms performed annually in the United States. The echocardiogram allows measurements of the size of the heart structures and the thickness of the heart muscle. The echocardiogram can be used assess the function and movement of the heart to identify tumors or emboli in the heart. In addition, the echocardiogram can also be used in the detection of structural abnormalities of the heart wall, the valves, and the blood vessels transporting blood to and from the heart. Interpretation of the echocardiogram can be used to diagnose congenital heart disease (i.e., ventricular septal defect), cardiomyopathies, and aneurysms.

Computer vision, as a branch of machine learning, has been proved as an advanced technique of automated image interpretation. Computers are trained to follow deep learning algorithms for mimicking human vision in aspects of classification, object detection and segmentation. The rapidly increasing computational power allows more data to be trained on more complex deep learning architectures in a shorter amount of time. Deep Learning based real-time image processing technique has been applied into many aspects in the real life, like safety control of automatic drive and face recognition on mobile devices. Accuracy is the most crucial factor of those applications comparing other criterions like the frame rate. However, echocardiogram interpretation in clinical practice needs an assurance that the data is collected under a relatively high frame rate about 50 fps, which is challenging in terms of real-time inference by deep learning model, especially take the data transmission time into account.

Recent studies using convolutional neural networks (CNN) and its derived structures to perform predictions of cardiac functions and segmentations of cardiac structures have shown promising results. These automated algorithms take a stack of echocardiography images or video streams as the input, these automated algorithms directly output the predictions of cardiac functions and segmentations of cardiac structures after doing the inference in a parametric black box. Though the CNN approaches are efficient and present output that can be interpreted easily, the approaches present two main limitations. First, the parametric black box operates with inaccessible [what does inaccessible mean?] parameters which makes it difficult to fine-tune the entire CNN approach. Without the help of anatomical landmarks and classical echocardiographic assessment methods, the root cause finding for biased predictions and estimations becomes arduous. Second, the input data for the CNN approaches generally have a restriction of minimum one heart cycle in timing length, which is difficult to implement in real time, taking the relatively heavy weight of the model into account. Accordingly, a need exists for an improved deep learning-based method for interpreting echocardiogram results without the previously stated limitations.

This disclosure presents a fully automated method that combining a deep learning U-Net model with classical echocardiographic assessment methods to predict a left ventricular (LV) ejection fraction in real time. The automated method was developed fully in accordance with the cardiologist workflow. The disclosed automated method has accessible outputs at each key step of the automated method. Additionally, the method utilizes adjustable parameters, which make it easy to fine-tune and visualize the results conveniently. The method first identifies two-halves LV segmentations from the inference of deep learning model. With the help of a predicted long axis from the two-halves LV segmentations and a single-plane algorithm, a LV volume estimate can be obtained without perceptible latencies. The end-diastolic and end-systolic states can be detected by comparing the LV volumes at different frames within a very short period of time such as a quarter of a heart cycle. With each observation of end-systolic state, an ejection fraction of one heart cycle is estimated. This estimation can be used to provide the caregiver with a recommended diagnosis of certain cardiac disorders such as ventricular fibrillation, ventricular tachycardia, atrial fibrillation and prolonged pauses or asystole. The disclosure also demonstrated that the inference of a disclosed light weight model with post processing can be finished in a short time frame that enables real-time processing, so that the implementation on a mobile device is also feasible for common use. Moreover, beat-to-beat assessments like arrhythmia and heart rate can be predicted by using the outputs from different nodes of the pipeline.

### SUMMARY OF THE INVENTION

In a first embodiment, a method of predicting left ventricular ejection fraction by processing echocardiogram data performed by software executed on a computer is provided. The method includes inputting echocardiogram data from an imaging device, identifying two-halves left ventricle segmentation based on the echocardiogram data for each time frame, estimating a left ventricular volume or area from the two-halves left ventricle segmentations for the each time frame, detecting end-diastolic states and end-systolic states by comparing the left ventricular volumes or area of the each time frames, and predicting a left ventricular ejection fraction based on said end-systolic states and end-diastolic states.

In a second embodiment, a method of predicting left ventricular ejection fraction by processing echocardiogram data performed by software executed on a computer system is provided. The method includes inputting echocardiogram data from an imaging device, identifying two-halves left ventricle segmentation based on the echocardiogram data for each time frame, estimating a left ventricular volume or area from the two-halves left ventricle segmentations for the each time frame, detecting end-diastolic states and end-systolic states by comparing the left ventricular volumes or area of the each time frames automatically with a moving window, and predicting a left ventricular ejection fraction based on said end-systolic states and end-diastolic states.

In a third embodiment, a system for predicting left ventricular ejection fraction by processing echocardiogram data performed by software executed on a computer is provided. The system includes an echocardiogram device for acquiring echocardiogram images from a patient, a computer for processing the echocardiogram images with a method for predicting the left ventricular ejection fraction, and a display screen to display the echocardiogram images and a heart condition diagnosis based on the left ventricular ejection fraction of the each time frames generated by the method. The method for predicting the left ventricular ejection fraction includes inputting the echocardiogram images acquired from echocardiogram device, identifying two-halves left ventricle segmentation based on the echocardiogram images for each time frame, estimating a left ventricular volume or area from the two-halves left ventricle segmentations for the each time frame, detecting end-diastolic states and end-systolic states by comparing the left ventricular volumes or area of the each time frames automatically with a moving window, and predicting a left ventricular ejection fraction based on said end-systolic states and end-diastolic states.

### DESCRIPTION THE DRAWINGS

The accompanying drawings, which are incorporated in and constitute a part of the specification, illustrate various example methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. Furthermore, elements may not be drawn to scale.

FIG. 1 shows a method for automated left ventricular ejection fraction prediction according to one illustrative embodiment of the invention.

FIG. 1A shows a system for implementing the method of FIG. 1.

FIG. 2 shows graphs depicting an automated moving window end-diastolic and end-systolic detection algorithm generated in accordance with the method of FIG. 1.

FIG. 3 illustrates an example graph of a histogram to depict a dice coefficient distribution of to assess the deep learning model performance test performed in accordance with the method of FIG. 1.

FIG. 4 illustrates an example visualization of output of an automated longitudinal line prediction test performed in accordance with the method of FIG. 1.

FIG. 5 illustrates an example graph of longitudinal line prediction evaluation performed in accordance with the method of FIG. 1.

FIG. 6 illustrates example graphs of ejection fraction estimation performance performed in accordance with the method of FIG. 1.

FIG. 7 illustrates example graphs for validation of an end-systolic and end-diastolic frame detection performed in accordance with the method of FIG. 1.

FIG. 8A illustrates an example graph of a speed test of alternative real time local-server and local machine approaches.

FIG. 8B illustrates an example schematic of a speed test of alternative real time local-machine approach.

FIG. 8C illustrates an example schematic of a speed test of alternative real time local-server approach.

### DETAILED DESCRIPTION OF THE INVENTION

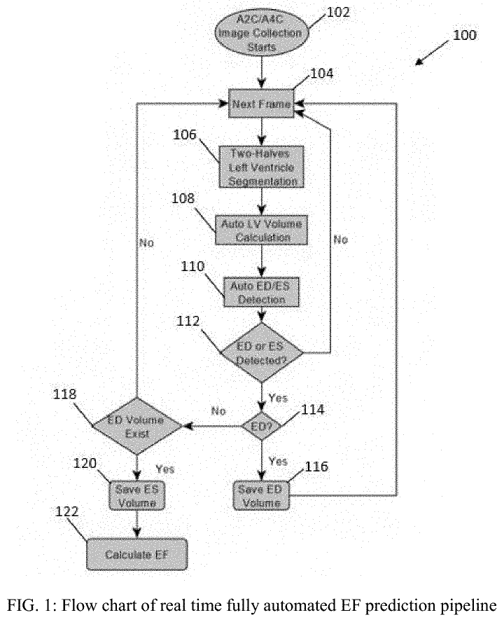

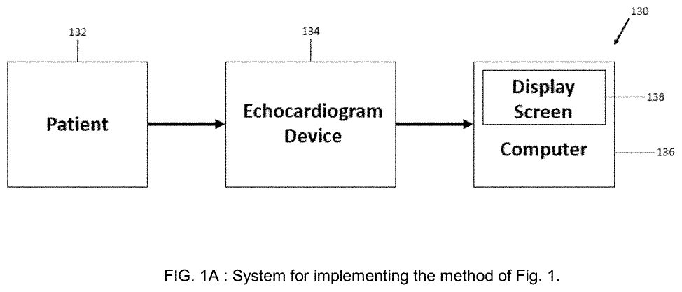

FIGS. 1 and 1A illustrate an example computerized method 100 and a system 130 for performing the method, respectively. The method 100 depicts an automated real time algorithm for estimating a left ventricular ejection fraction (EF). The computerized method 100 estimates the estimated EF in one heart cycle with real-time inputs through the detection of variables such as end-diastolic states (ED) and end-systolic states (ES) for each heart cycle. With the estimation of EF for one heart cycle, the computerized method 100 of FIG. 1 proceeds to estimate the EF of the next heart cycle. The EF of multiple heart cycles can be averaged to present an estimated average EF. The method can be performed by a computer 136 connected to an echocardiogram device 134 used to take echocardiogram images from a patient 132. The computer includes a display screen 138 to display the echocardiogram images, as well as a heart condition diagnosis based on the predicted EF generated by the method. Preferably, the method will generate a recommended diagnosis for display to a caregiver at the point of care, or remotely.

The computerized method 100 was created with a U-Net architecture based deep-learning model, which is widely used for semantic segmentation tasks of medical images. The U-Net architecture consists of an encoder and decoder. The encoder by is formed by picking some intermediate layers of a pre-trained MobileNetV2 model and the decoder is formed by a series of up-sample blocks. A dropout has been applied to prevent overfitting in the encoder and decoder. Both encoder and decoder were trained during the training process. Input and output layers of the computerized method 100 have a matrix size of 128*128*3 since there are three possible output labels for each pixel. In one embodiment, the computerized method 100 of FIG. 1 has 6,504,227 parameters, with 6,471,395 parameters that are trainable among them. The computerized method 100 of FIG. 1 is compiled with an Adam optimizer. A loss function was used in the computerized method 100 of FIG. 1 to reduce computational cost. In one embodiment, a sparse categorical cross entropy (SCCE) was chosen as the loss function in light of computer cost efficiency and need for working more than a project with two classes. Ten thousand images were extracted from a Stanford Dataset, which contains 10300 A4C echocardiogram videos with expert annotations and labels. The images were used to train the computerized method 100 of FIG. 1 and a 10-fold cross validation method has been applied to evaluate model performance during training. Early stopping was used to reduce the training time by setting a patience value of 10.

The computerized method 100 includes, at 102 and 104 of FIG. 1, inputting echocardiogram data from an imaging device. Echocardiogram data is collected to provide images 102 for the computerized method 100 in FIG. 1. The images are typically apical-2-chambers (A2C) or apical-4-chambers (A4C). In one embodiment, to provide images 102 for the computerized method 100 in FIG. 1, the images are extracted from the Stanford dataset, which contains 10300 A4C echocardiogram videos with expert annotations and labels. The echocardiogram videos have pixel-wise resolution of 112*112. Most The echocardiogram videos have frame rates between 30 to 80 frames per second (FPS), with most echocardiogram videos having a frame rate of 50 FPS. Each echocardiogram video includes at least one complete heart cycle, with as many as 20 complete heart cycle. A frame 104 from the images 102 extracted from the echocardiogram videos was provided as the input to the computerized method 100 in FIG. 1. The computerized method 100 provided the EF for each frame 104 in a cyclical fashion.

The computerized method 100 further includes, at 106 of FIG. 1, identifying two halves left-ventricle segmentation based on the frame 104 of the images 102 extracted from the echocardiogram videos. In one embodiment, a boundary is estimated with a two halves left ventricle mask with three labels (background, left, and right half of left ventricle), which can detected by passing a 2*2 averaging filter to the input frame 104 and getting pixels with the intensity that is equal to an average of label intensities besides background. A longitudinal line is derived from connecting points with a maximum and minimum y values, which are treated as the top and bottom points of the longitudinal line. The identification of two halves left-ventricle segmentation 106 also comprises an algorithm to detect improper positioning of the boundary line. A pre-defined threshold is set for distance between two adjacent points. The identification of two halves left-ventricle segmentation 106 is further programmed to delete points if the distance between the two adjacent points is over the threshold until five consecutive distances are all within the pre-defined threshold. A volume of the LV 108 is calculated by calculating volumes of each disks for each point on the longitudinal line. In one embodiment, a volume of the each of the disk is calculated by treating that the disk is a cylinder. A diameter of the disk is determined by calculating a distance between intersection points between a line perpendicular to the longitudinal line and boundary line of the left ventricle mask. A radius is calculated as half the value of the diameter. A height of the disk is determined by calculating distance between two adjacent points of the longitudinal line. In another embodiment, the disks are treated as a circular truncated cone to improve accuracy for high-resolution inputs. The volume of each disk is defined as:

$$V = π {r}_{0}^{2} d + π ( \frac{{r}_{1}^{2}}{3} - \frac{{r}_{0}^{2}}{6} - \frac{{r}_{0} {r}_{1}}{6} ) d$$

where r₀ and r₁ are the radiuses of two adjacent disks, d is the distance between two adjacent disks.

The computerized method 100 further comprises automatic detection of ED or ES 110 determined from the volume of the LV 108. In one embodiment, if an instance if the ED or ES is not detected 112, the computerized method 100 of FIG. 1 proceeds to restart with the next frame 104. In an instance the ED is detected 114, a volume of ED is saved 116 by the computerized method 100. In the instance the ED is not detected, the computerized method 100 of FIG. 1 confirms if the ED has already been detected in the frame and confirm that the ES is detected 118 or restart the computerized method 100 of FIG. 1. If the ES is confirmed 118, a volume of ES is saved 120 by the computerized method 100. In one embodiment, the estimated of the EF 122 is derived from the volume of ED saved 116 and the volume of ES saved 120 with the formula:

$$E F = \frac{{V}_{E D} - {V}_{E S}}{{V}_{E D}}$$

where V_{ED} and V_{ES} are the volumes of ED and ES respectively and EF is the LV ejection fraction.

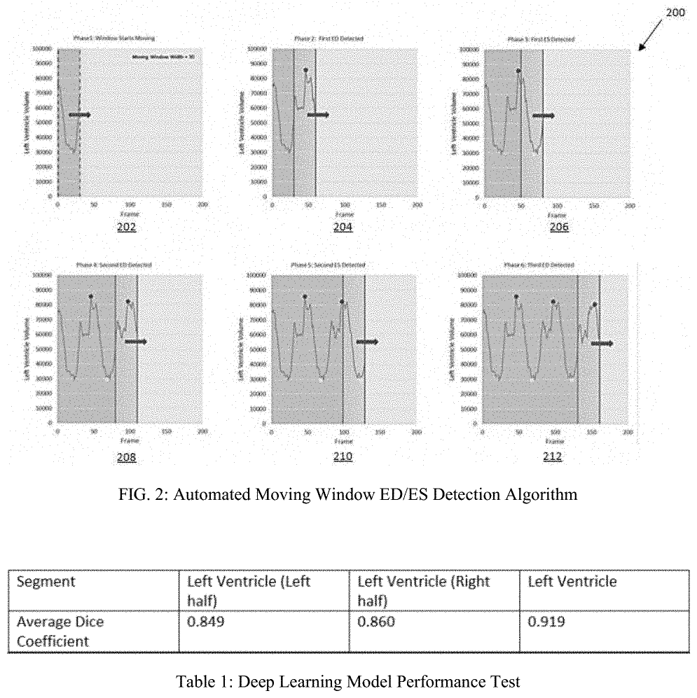

FIG. 2 illustrates exemplary graphs depicting an automated moving window 200 used for the automatic detection of ED and ES 110 determined from the volume of LV 108 as determined by the computerized method 100 of FIG. 1. With the volume of LV 108, an adjustable moving window 202 is initiated to detect a first ED 204 and a first ES 206. The adjustable moving window 202 is used to compute a second ED 208 and a second ES 210 from the volume of LV 108 of the next frame 104. The adjustable moving window 202 continues the automatic detection ED and ES 110 (e.g., a third ED 212) determined from the values of volume of LV 108 as determined by the computerized method 100 of FIG. 1 to estimate the EF 122. The adjustable moving window 202 is calculated by verifying if

$$\vert {V}_{w} - {V}_{1} \vert < α \times \frac{{\sum }_{i = 1}^{w - 1} \vert {V}_{i + 1} - {V}_{i} \vert}{w - 1}$$

applies in the adjustable moving window 202 and

$$\{ \begin{matrix}\begin{matrix}ED position in the window = \\ arg max \{ {V}_{1} , … , {V}_{w} \} ,\end{matrix} & if {\sum }_{i = 1}^{\frac{w}{2}} ( {V}_{i + 1} - {V}_{i} ) > 0 \\ \begin{matrix}ES position in the window = \\ arg min \{ {V}_{1} , … , {V}_{w} \} ,\end{matrix} & if {\sum }_{i = 1}^{\frac{w}{2}} ( {V}_{i + 1} - {V}_{i} ) < 0\end{matrix}$$

Where vᵢ is the ith volume in the window, w is the width of the window, and α is a parameter to adjust the sensitivity of the algorithm. α is adjusted to prevent false positives and false negatives. α and w should be determined by the input frame 104. In this case, w=30, α=1.5.

### Results

### Deep Learning Model Performance

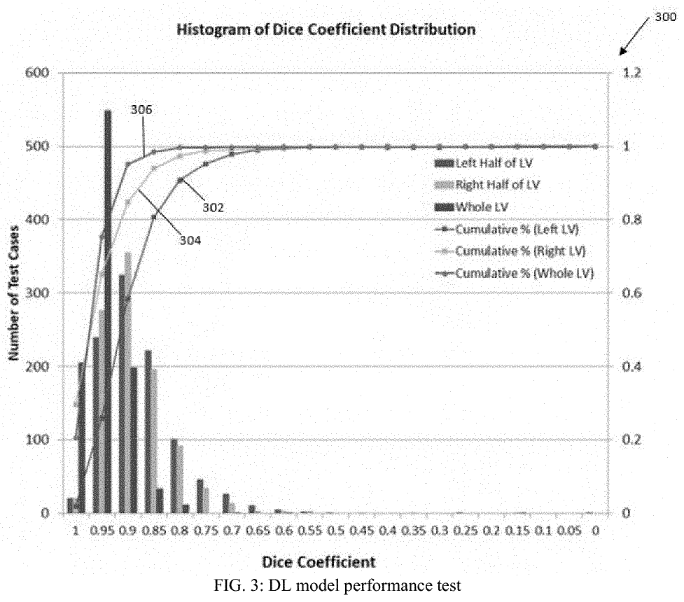

In reference to FIG. 3, an exemplary graph 300 of a histogram depicting a dice coefficient distribution is presented to assess a performance of the computerized method 100 of FIG. 1. To ensure the accuracy of the computerized method 100 of FIG. 1 in identifying the two halves left-ventricle segmentation based on the frame 104 of the images 102 extracted from the echocardiogram videos, a dice coefficient is calculated for an input of 1000 A4C images extracted from echocardiogram videos 102 in the computerized method 100 of FIG. 1. as a cumulative percentage for a left half mask of the LV 302, a right half mask of the LV 304, and a merged mask of the whole LV 306, produced by combining the left half mask of the LV 302 and the right half mask of the LV 304. The dice coefficients are presented in Table 1 the merged mask of the whole LV 306, the left half mask of the LV 302 and the right half mask of the LV 304 are presented, with the merged mask of the whole LV 306 having a score of 0.919.

TABLE 1

| | Left Ventricle | Left Ventricle | |
|---|---|---|---|
| Segment | Left half) | (Right half) | Left Ventricle |
| Average Dice | 0.849 | 0.860 | 0.919 |
| Coefficient | | | |

### Performance of Longitudinal Line Prediction

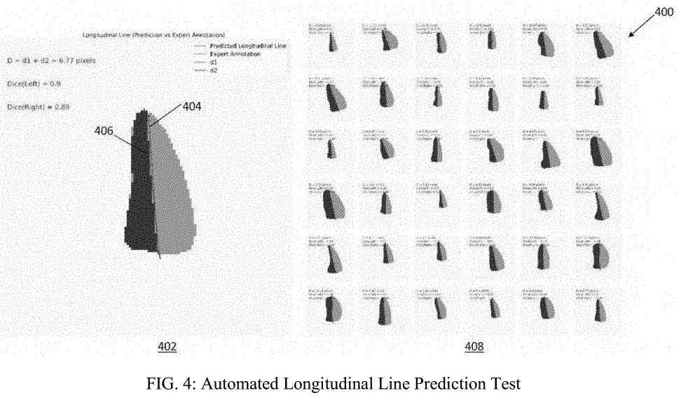

In reference to FIG. 4, exemplary visualizations 400 of output of the automated longitudinal line as part of the identifying two halves left-ventricle segmentation based on the frame 104 of the images 102 extracted from the echocardiogram videos are shown. The example visualizations 400 are extracted from the input of 1000 A4C images extracted from echocardiogram videos 102 in the computerized method 100 of FIG. 1. 37 cases from the test data were selected to predict the longitudinal line. A shift distance, D (pixels), is calculated between an expert predicted longitudinal line 406 and an automated longitudinal line 404 predicted by the computerized method 100 of FIG. 1 for an example visualization on a single case 402 by summation of translations of top and bottom longitudinal vertexes:

D(pixels)=d₁+d₂

Where d₁ and d₂ are the apex and mid-base translations between expert annotation and prediction of the computerized method 100.

The discrepancy, D (pixels) was calculated for examples visualizations of remaining 36 cases 408. Values of D was smaller than 7 pixels from the examples visualizations 402 and 408.

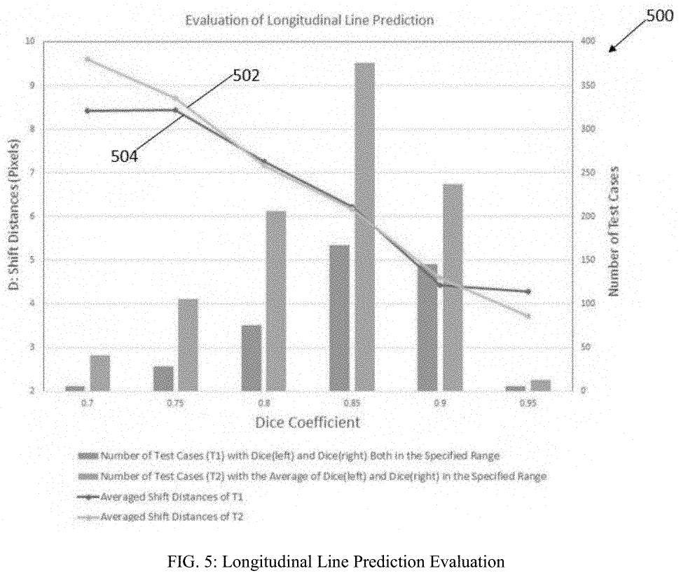

FIG. 5 presents a graph 500 of longitudinal line prediction evaluation. The input of 1000 A4C images extracted from echocardiogram videos 102 in the computerized method 100 of FIG. 1 are separated into two groups, T1 an T2. Group T1 is prepared by dividing the input of 1000 A4C images by limiting dice coefficients derived from left half of LV determined by the left half mask of the LV 302 and right half of LV determined by the right half mask of the LV 304 in a pre-defined dice coefficient interval. Group T2 is prepared by dividing the input of 1000 A4C images by limiting averaged dice coefficients derived from left half of LV determined by the left half mask of the LV 302 and right half of LV determined by the right half mask of the LV 304 in a pre-defined interval as:

$$T ∈ \{ \begin{matrix}{T}_{1} , & if ( {S}_{1} \leq {Dice}_{{T}_{left}} < {S}_{2} ) ⩓ ( {S}_{1} \leq {Dice}_{{T}_{right}} < {S}_{2} ) \\ {T}_{2} , & if {S}_{1} \leq \frac{{Dice}_{{T}_{left}} + {Dice}_{{T}_{Right}}}{2} < {S}_{2}\end{matrix}$$

Where variable S₁ is set from 0.7 to 0.95 with 0.05 as a step and variable S₂=S₁+0.05.

Shift distances for group T1 504 and shift distances for group T2 502 are determined in FIG. 5. Shift distances for group T1 504 and shift distances for group T2 502 both show a descending trend, which is an indication that a good segmentation result is the key factor of a decent longitudinal line prediction. Moreover, Shift distances for group T1 504 and shift distances for group T2 502 show that groups T1 and T2 exhibit similar results on mid-range dice coefficients but group T2 performs worse on a low dice coefficient range and better on a high dice coefficient range. FIG. 5 further illustrates that worse segmentation between left and right halves of LV dominates the longitudinal line predication on low dice coefficient cases, whereas the better segmented half dominates the longitudinal line prediction on high dice coefficient cases.

### Ejection Fraction Estimation

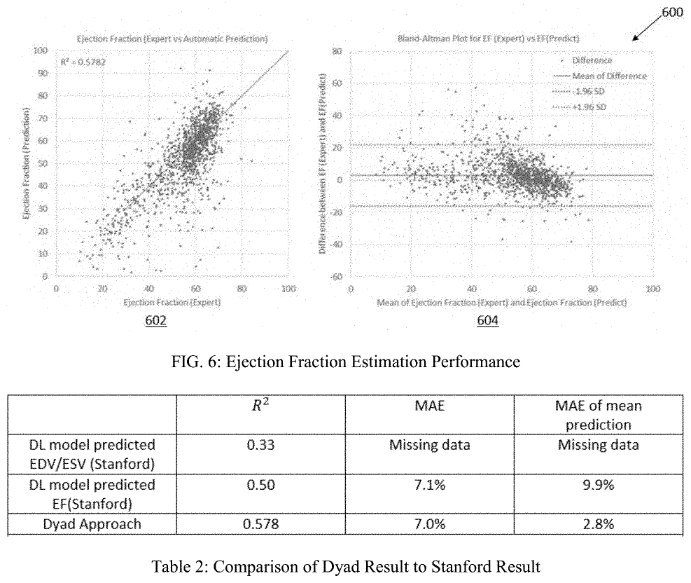

FIG. 6 illustrates example graphs of ejection fraction estimation performance. In the computerized method 100 of FIG. 1, the EF is estimated 122 from the volume of ED saved 116 and the volume of ES saved 120. To ensure the accuracy of the EF estimation 122, the EF is estimated from 1277 sets of ED and ES images that are labeled as test group in the Stanford dataset with both the computerized method 100 of FIG. 1 and manual calculation by experts. A linear regression analysis graph 602 and a Bland-Altman plot of EF 604 are used to derive a R² score of 0.578 and a mean absolute Error (MAE) of 7.1% and MAE of mean prediction of 2.8%, as shown in Table 2.

TABLE 2

| | | | MAE of mean |
|---|---|---|---|
| | R² | MAE | prediction |
| DL model predicted | 0.33 | Missing data | Missing data |
| EDV/ESV (Stanford) | | | |
| DL model predicted | 0.50 | 7.1% | 9.9% |
| EF(Stanford) | | | |
| Dyad Approach | 0.578 | 7.0% | 2.8% |

### ES/ED Frame Detection in Real-Time

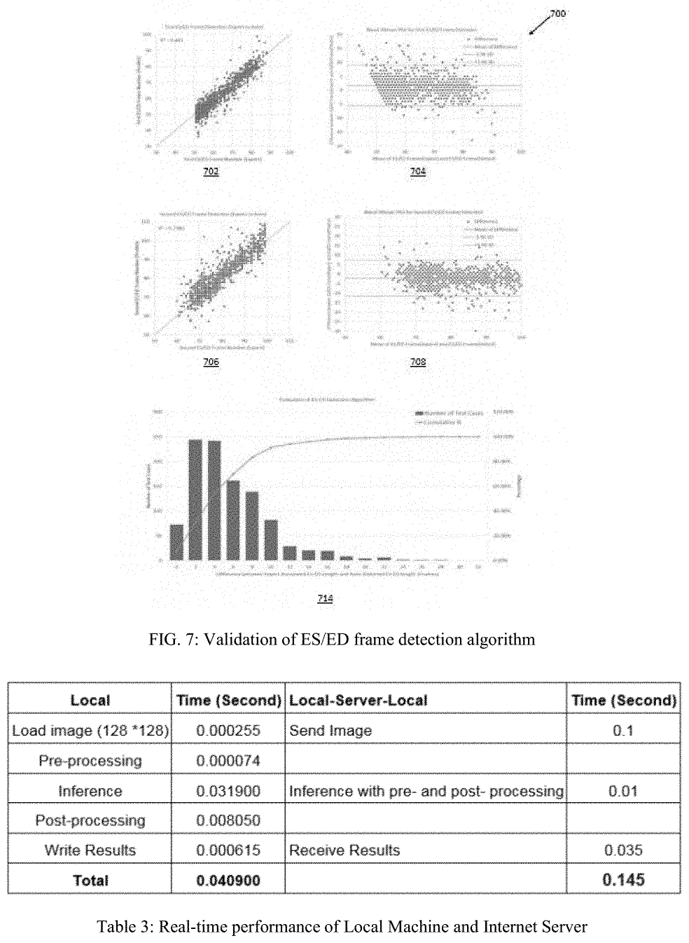

FIG. 7 presents example graphs 700 for validation of the automatic ES and ED detection 110 generated by method 100 of FIG. 1. To compare the ES and ED frames estimated by automatic ES and ED detection algorithm 110 and predicted by the Stanford Dataset, graphs of linear regression analysis to calculate the R² (denoted by example linear regression graphs of a first ED/ES frame detection 702 and a second ED/ES frame detection 706) and Bland-Altman plots of ES and ED frame detection (denoted by example Bland-Altman plots of a first ED/ES frame detection 704 and a second ED/ES frame detection 708) were derived. In order to validate the automated ES and ED detection 110 as presented by the computerized method 100 of FIG. 1 with the expert labeled ED and ES frames in real-time, the following criteria was applied:

1. Only use the videos that have annotated ES and ED frame numbers both between 50 and 100.

2. Finding the frame number in the detected ES/ED list that has the smallest absolute error with the smaller frame number in the expert defined ES/ED list.

3. Making the two-frame ES/ED list by using the frame that is found in step 2 and its next element in the prediction list. Then test the performance of algorithm of the computerized method 100 of FIG. 1 by comparing the two-frame ES/ED list with the expert training dataset defined ES/ED list.

The automatic ES and ED detection 110 as presented by the computerized method 100 of FIG. 1 was able to precisely detect the first and second ES/ED frame with a R² value of 0.88 and 0.80, and a mean of difference of 1.66 and −2.14 frames respectively. FIG. 7 further includes a histogram 710 in which the number of frames between ES and ED predicted by expert annotations and the automatic ES and ED detection 110 as presented by the computerized method 100 of FIG. 1 is subtracted for each case. About 80% of the cases are with the discrepancy smaller than 8 frames, a high sensitivity in terms of detecting the global minimum/maximum left ventricular volumes in a half heart cycle and reliability of the automatic ES and ED detection 110 as presented by the computerized method 100 of FIG. 1.

### Real-Time Performance Evaluation

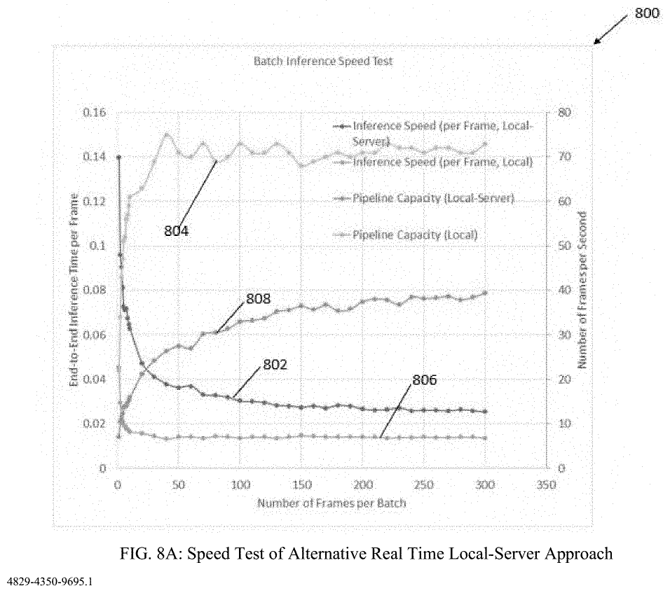

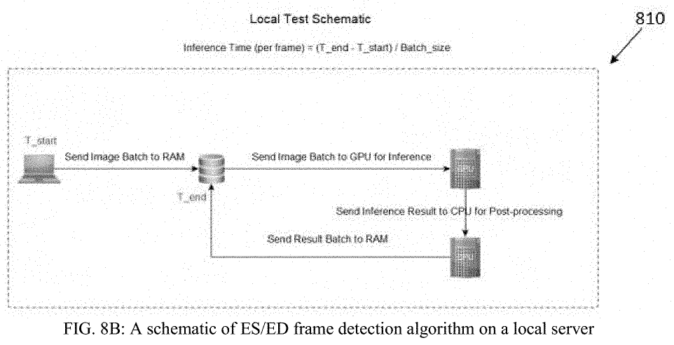

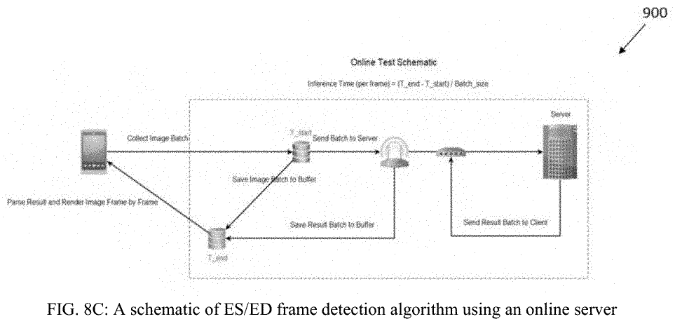

FIG. 8A illustrates an example graph 800 of a speed test of alternative real time local-machine and online server approaches in which an inference speed 806 and a pipeline capacity 804 with the local machine approach as well an inference speed 802 and pipeline capacity 808 of the online serve approach are calculated.

To evaluate the computerized method 100 of FIG. 1, efficient beat-to-beat analysis, usually the limitation in the process of data transmission and deep learning inference, was used as a metric. The computerized method 100 of FIG. 1 is tested with a local machine, i7-7700 CPU, DDR4 RAM and Nvidia GeForce 2080 Super Graphic Card, as evidenced by a schematic 810 in FIG. 8B. An average end-to-end processing time is estimated to be about 0.04 seconds, as shown in Table 3 (result of processing one image at a time with the computerized method 100 of FIG. 1), translating to 25 frames per second. It is further deduced that in the computerized method 100 the data transmission takes a very short amount of time that is almost neglected, whereas 80 percent of the time is spent on the deep learning inference.

TABLE 3

| | Time | | Time |
|---|---|---|---|
| Local | (Second) | Local-Server-Local | (Second) |
| Load image (128 *128) | 0.000255 | Send Image | 0.1 |
| Pre-processing | 0.000074 | | |
| Inference | 0.031900 | Inference with pre- | 0.01 |
| | | and post- processing | |
| Post-processing | 0.008050 | | |
| Write Results | 0.000615 | Receive Results | 0.035 |
| Total | 0.040900 | | 0.145 |

To evaluate the computerized method 100 of FIG. 1 through an online test, a Tensor RT Inference Server provided by Nvidia was set up, one frame at a time was sent from the local machine to the online server and one cycle was completed after receiving the result file from the server. This approach reduced two-third of the deep-learning model inference time, which is more than enough of catching up the frame rate during data collection. However, the real-time data capacity is still constrained by the time cost of data transmission and HTTP request.

To overcome the limitation of the cost of data transmission and HTTP requests, the computerized method 100 of FIG. 1 was tested by reducing the number of HTTP requests, as in the case of the state-of-the-art development of mobile network. Instead of sending one image and receiving one result at a time, the computerized method 100 of FIG. 1 was set up by sending batches of sequenced images to the server and performing the inferences of the batch as evidenced in a schematic 820 of FIG. 8C. The outputs are also sent from the server as a batch. Batch processing improved the operation of the system considerably. With the setup of the number of images in each batch from 1 to 300, the framework is able to process more than 35 frames with the batch size of 150 as shown in the example graph 800 of a speed test of alternative real time local-server approach.

The computerized method 100 depicted in FIG. 1 presents a novel approach of fully automatically evaluating LV EF in real time by combining the classical cardiac theories with deep learning semantic segmentation models. Taking the attributes of accuracy and efficiency that is essential for real-time analysis of cardiac functions into account, the computerized method 100 is proved to be able to operate the input frames with high frequency to obtain necessary information for cardiac beat-to-beat analysis with the accuracy that is competent for portable preliminary diagnosis purpose.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method of predicting left ventricular ejection fraction by processing echocardiogram data performed by software executed on a computer, the method comprising:
inputting echocardiogram data from an imaging device;
identifying two-halves left ventricle segmentation based on the echocardiogram data for each time frame;
estimating a left ventricular volume or area from the two-halves left ventricle segmentations for the each time frame;
detecting end-diastolic states and end-systolic states by comparing the left ventricular volumes or area of the each time frames; and
predicting a left ventricular ejection fraction based on said end-systolic states and end-diastolic states.

2. The method according to claim 1, wherein estimating the left ventricular volume from the two-halves left ventricle segmentations comprises:
estimating a longitudinal line from the two-halves left ventricle segmentations for the each time frame.

3. The method of according to claim 2, wherein the longitudinal line can be estimated by connecting a top point and bottom point of a boundary created by a left half ventricle mask and a right half left ventricle mask.

4. The method according to claim 1, wherein the detection of the end-diastolic states and the end-systolic states is done annotated manually.

5. The method according to claim 1, wherein the detection of the end-diastolic states and end-systolic states is done automatically by a moving window approach.

6. The method according to claim 1, wherein the input echocardiogram data is real-time.

7. The method according to claim 1, wherein the left ventricular volume is estimated for the each time frame within a short period of time.

8. The method according to claim 7, wherein the short period of time is a quarter of a heart cycle.

9. The method of according to claim 1, further comprising detection of a cardiac anomaly.

10. The method according to claim 1, wherein the left ventricular ejection fraction is predicted by the estimated volume of end-diastolic and end-systolic states.

11. The method according to claim 1, wherein the left ventricular ejection fraction is predicted by a deep learning model using end-diastolic and end-systolic images.

12. The method according to claim 5, wherein a sensitivity of the moving window is determined by a frame rate of the echocardiogram data.

13. A method of predicting left ventricular ejection fraction by processing echocardiogram data performed by software executed on a computer system, the method comprising:
inputting echocardiogram data from an imaging device;
identifying two-halves left ventricle segmentation based on the echocardiogram data for each time frame;
estimating a left ventricular volume or area from the two-halves left ventricle segmentations for the each time frame;
detecting end-diastolic states and end-systolic states by comparing the left ventricular volumes or area of the each time frames automatically with a moving window; and
predicting a left ventricular ejection fraction based on said end-systolic states and end-diastolic states.

14. A system for predicting left ventricular ejection fraction by processing echocardiogram data performed by software executed on a computer, the system comprising:
an echocardiogram device for acquiring echocardiogram images from a patient;
a computer for processing the echocardiogram images with a method for predicting the left ventricular ejection fraction, the method comprising:
inputting the echocardiogram images acquired from echocardiogram device;
identifying two-halves left ventricle segmentation based on the echocardiogram images for each time frame;
estimating a left ventricular volume or area from the two-halves left ventricle segmentations for the each time frame;
detecting end-diastolic states and end-systolic states by comparing the left ventricular volumes or area of the each time frames automatically with a moving window; and
predicting a left ventricular ejection fraction based on said end-systolic states and end-diastolic states; and
a display screen to display the echocardiogram images and a heart condition diagnosis based on the left ventricular ejection fraction of the each time frames generated by the method.
